(19) United States
(12) Patent Application Publication
(10) Pub. No.: US 2004/0267262 A1
(43) Pub. Date: Dec. 30, 2004

(54) Pedicular screw arrangement

(76) Inventor: Helmut D. Link, Hamburg (DE)

Correspondence Address: MORRISON & FOERSTER LLP, 1650 TYSONS BOULEVARD, SUITE 300, MCLEAN, VA, 22102, US

(21) Appl. No.: 10/483,120

(22) PCT Filed: Jul. 10, 2002
(86) PCT No.: PCT/EP02/07705

(30) Foreign Application Priority Data
Jul. 10, 2001 (EP) 01116869.7

(51) Int. Cl. A61B 17/56

(52) U.S. Cl. 606/72

(57) ABSTRACT

Pedicular screw arrangement with a pedicular screw support (4). The latter has a bore hole (10) cooperating with the pedicular screw (1), and one or more supporting surfaces (7) which rest on an appropriate bone surface, in particular on the surface (3) of the saddle which is located between the costal process and the superior articular process (2).

For stabilizing, alignment or fusion of vertebral bodies, appliances are used which consist of pedicular screws and of rods connecting these pedicular screws. The pedicular screws are screwed through the pedicle into the vertebral body between the costal process and the superior articular process. The pedicle is extremely strong and is able to absorb the forces transmitted by the pedicular screws. Sometimes the screws fracture because of the substantial forces and in particular the bending moments which act on the pedicular screws.

This can be avoided (U.S. Pat. No. 5,733,284) by a system in which the forces originating from the rods are transmitted to the bone not via the pedicular screws, but via shaped pieces which are adapted to the bone and have supporting surfaces which grip like hooks from below or above on parts of the vertebral body. The pedicular screws then serve only to secure these shaped pieces on the bone, without having to transmit vertical forces. However, this system is extremely complex in design and in operative use. Moreover, it is only effective if there is almost perfect adaptation to the bone, which is not really feasible in practice.

Moreover, shaped pieces are known for connecting longitudinally extending rods to a number of vertebral bodies (U.S. Pat. No. 4,697,582, U.S. Pat. No. 6,136,002, U.S. Pat. No. 4,697,582, U.S. Pat. No. 6,136,002, U.S. Pat. No. 4,289,123), which shaped pieces are not specifically provided for pedicular screws and also have no supporting surfaces engaging a bone from above or below.

The invention therefore starts out from the cited prior art in which the forces acting parallel to the spinal column direction are transmitted principally via the pedicular screws. The object of the invention is to improve the force transmission in the area of the screws. This object is achieved by the features of claim 1 and, preferably, the features of the dependent claims.

According to the invention, the pedicular screw arrangement comprises a pedicular screw support. The latter has, on the one hand, a bore hole adapted to the shank of the pedicular screw and tightly enclosing and supporting the screw shank. On the other hand, it comprises a shaped piece which has at least one supporting surface for bearing on the bone from above and/or below. To ensure that this can transmit some of the forces in question from the screw to the bone, it extends basically parallel to the screw direction. This means that, in sagittal section, it must enclose, with the direction of the bore hole (which is also the screw direction), an angle which is sufficiently small, preferably less than 40°. It preferably has a part in which this angle is less than 20°, more preferably less than 10°. The support is preferably above or below the entry point of the screw on the saddle surface between the superior articular process and the costal process. Other suitable sites for support are the underside or top side of the costal process or the underside of the superior articular process. Support from above on the superior articular process is also conceivable.

The bore hole in the pedicular screw support should be of such a length that it substantially guarantees direction equivalence between the screw and the bore hole. It is particularly advantageous if it is formed by a tube part which is connected rigidly to the shaped piece forming the supporting surfaces. This tube part can protrude from the shaped piece away from the bone in the direction of the screw head, if there is enough space there for supporting the screw shank at this point and strengthening it against bending stresses. The lever arm of the bending moment acting on the screw shank is shortened in this way and the load exerted on the screw is reduced.

Instead of this, or in addition, provision can be made that a tube part involved in the formation of the bore hole protrudes on the other side from the shaped piece forming the supporting surface(s). Since the shaped piece normally lies on the bone surface in the area of the entry point of the screw, this tube part extends into the bone. To receive it, the bone is reamed out or drilled slightly more than is customary for receiving only the screw shank. The greater diameter of the tube part compared to the screw shank affords a correspondingly larger force transmission surface toward the bone. In addition to the supporting surfaces resting on the bone from above or from below, the risk of the screw shifting and deforming the bone under high loads is also reduced. In addition, the distance within which the screw shank finds a support reducing the load upon it is increased. The tube piece protruding on the side toward the bone and extending into the bone is expediently at least partially conical on the outside.

According to a further feature of the invention, it should be of such a length that it protrudes at least as far as the supporting surface. This has the advantage that the force transmission from the screw shank to the pedicular screw support and from the supporting surfaces thereof to the bone does not lead to a bending moment which could tend to shift the position of the pedicular screw support. Advantageously, this tube part is still longer, namely with a total length, to be accommodated in the bone, of the order of 1 to 1.5 cm.

If the implant is a permanent one, it may be expedient to configure the outside of the tube part in such a way that an intimate contact with the bone is favored. This can be done, for example, by means of a suitable porous surface or a growth-promoting coating.

The fit of the shaped piece forming the supporting surface(s) on the bone can if appropriate be improved by teeth or screws protruding into the bone.

It is generally not necessary for the supporting surfaces, after implantation, to rest directly and without clearance on the bone surface. Instead, it suffices in most cases if they are at a distance from the bone surface small enough to allow them to exert their supporting action should the screw shank shift in the bone under the effect of forces.

Normally, the pedicular screw support is assigned in each case to only one pedicular screw. This allows it to be kept relatively small, which makes its operative use easier and minimizes the possibility of inadequate adaptation to the bone shape; the number of sizes to be made available can be correspondingly small.

The invention is explained in greater detail below with reference to the drawing, in which:

FIG. 1 shows a sagittal section through the pedicular screw entry point in a first embodiment,

FIG. 2 shows a dorsal view in the direction of the pedicular screw axis,

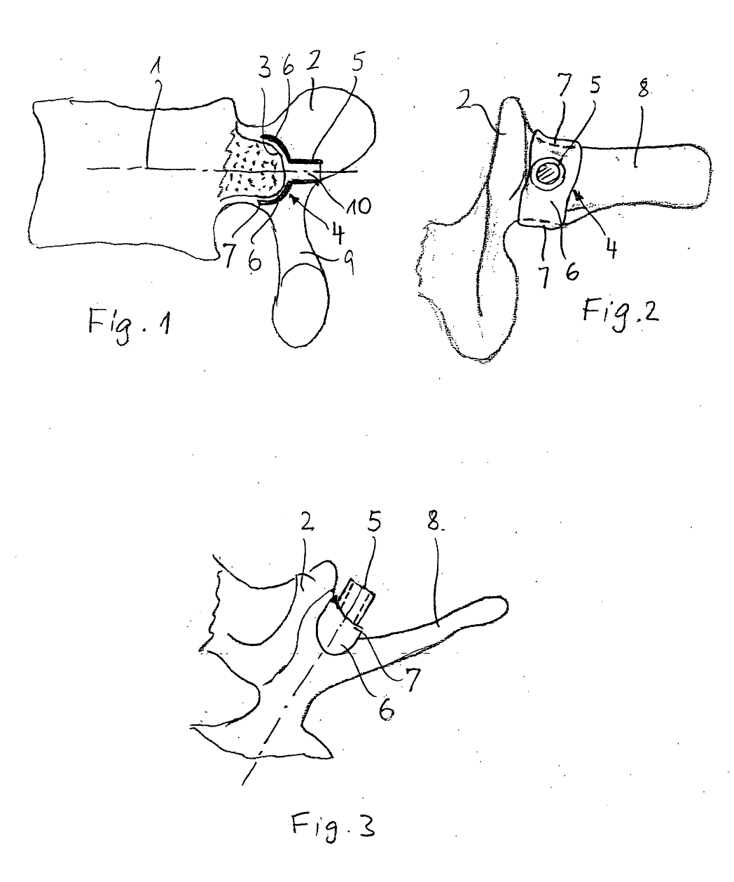

FIG. 3 shows a view of this region from above,

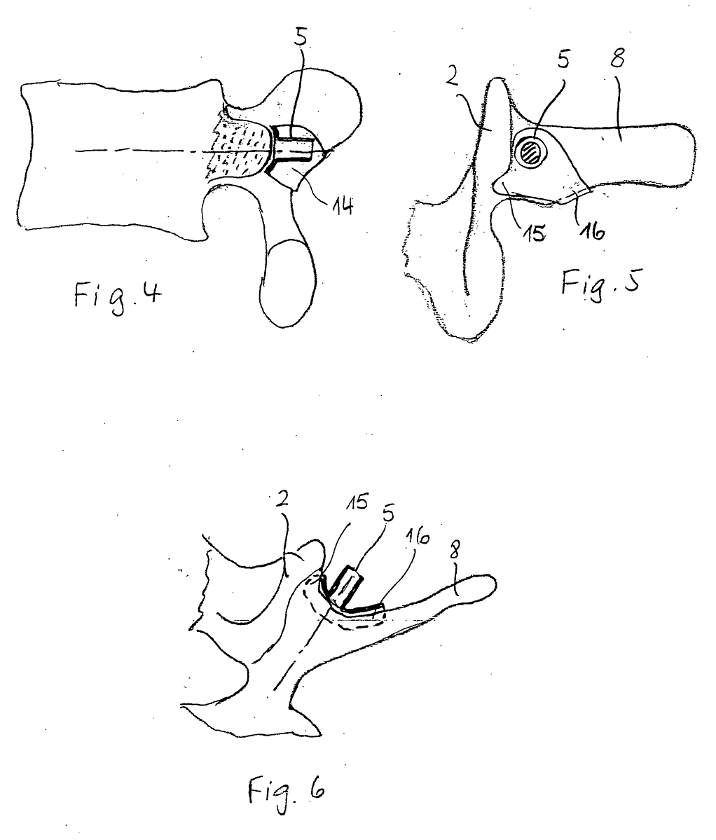

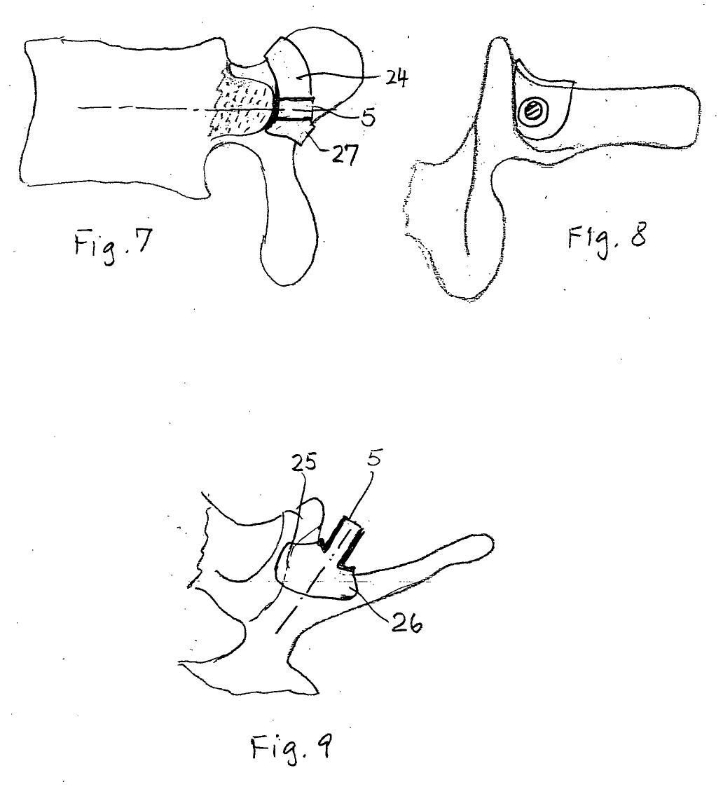

FIGS. 4-6 show corresponding views of a second embodiment,

FIGS. 7-9 show corresponding views of a third embodiment, and

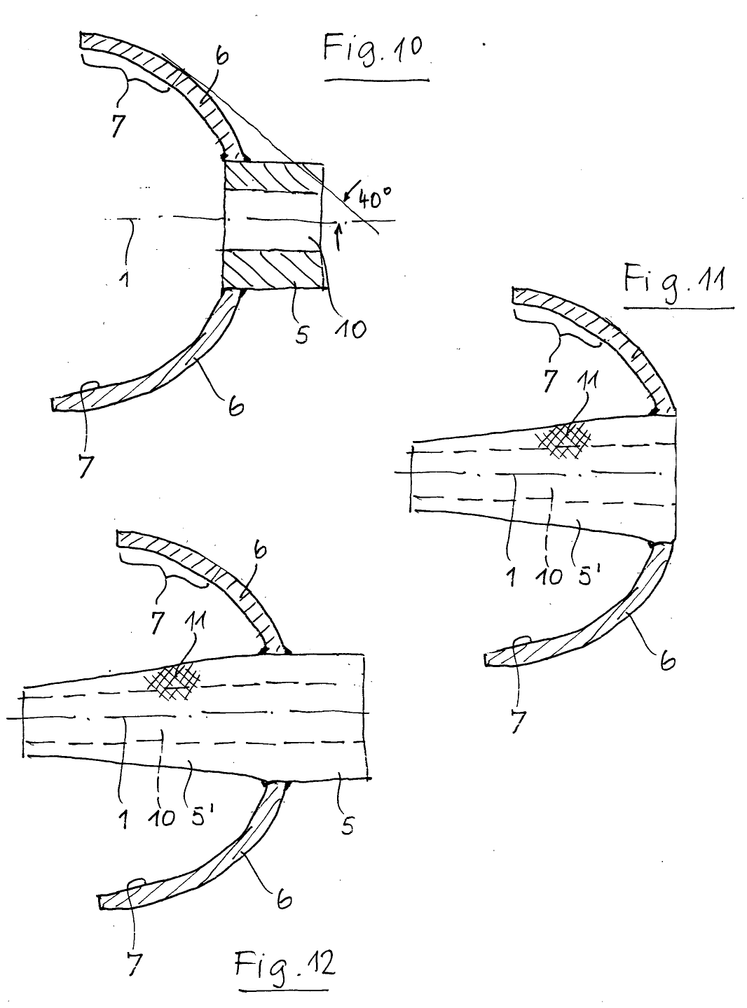

FIGS. 10-12 show enlarged views of alternative embodiments of the pedicular screw support.

In FIG. 1, a pedicular screw is represented by its axis 1, shown as a dot-and-dash line. Between the superior articular process 2 and the costal process lying in front of the plane of the drawing, it enters the surface of the bone of the saddle 3 between these two processes.

The pedicular screw support 4 has a tube part 5 for supporting the screw shank. A sufficiently tight fit ensures that the support 4 has to take part in any movements of the screw shank or, to put it the other way, the screw shank bears on the support if the latter cannot follow its movement tendencies.

The support 4 comprises a shaped piece which is connected rigidly to the tube part 5 and which consists of wings 6 which are curved to match the saddle surface 3 and rest on the saddle surface 3. They enclose the latter to such an extent that their end areas 7 approximate to a direction parallel to the direction 1 of the screw shank by up to less than 40°, preferably less than 20°, preferably less than 100. These end areas form the support surfaces 7. Since they extend substantially parallel to the screw direction 1, they are able to support the screw upon transmission of transverse forces to the bone. The wings 6 can be provided with teeth or screws which penetrate into the saddle surface 3 in order to additionally stabilize the pedicular screw support 4 on the bone. It can be seen particularly clearly from FIG. 1 how the wings rest with their supporting surfaces 7 on the saddle surface from above and from below, said saddle surface lying between the superior articular process 2 and the costal process 8. The same can be seen from the plan view in FIG. 3 in respect of the wing 6.

Details of the pedicular screw support 4 emerge from the enlarged view in FIG. 10. To receive the pedicular screw, it has a tube part 5 protruding from the convex side of the shaped piece 6 directed away from the bone.

The bore hole 10 inside the tube part 5 is cylindrical and corresponds, with slight play, to the external diameter of the pedicular screw (not shown). The pedicular screw support 4 is therefore always oriented in the same direction as the pedicular screw, with a common center line 1.

The angle which the wings 6 enclose at each point, in sagittal section, with the direction 1 decreases toward the wing ends, until it falls below the 40° figure. According to the definition of claim 1, the supporting surfaces 7 then begin.

The angle of 40° is randomly set. It is appreciated that the ability of the supporting surfaces to transmit forces extending transverse to the pedicular screw is better, the smaller the angle they enclose with the axis direction 1, so that it seems undesirable to choose angles that are too great. However, the invention does not exclude the possibility that the support effect according to the invention can also be achieved when the angle is greater than 40°, depending on the overall technical picture. This claim feature is accordingly to be interpreted taking into consideration the effect achieved.

The second illustrative embodiment according to FIGS. 4 through 6 shows that the tube part 5, which is intended to tightly surround the screw shank, is connected rigidly to a shaped metal piece 14 which with a first wing 15 engages under the superior articular process and with a second wing 16 engages under the costal process. Forces tending to lift the screw shank are thus safely transmitted to said bone surfaces.

The illustrative embodiment according to FIGS. 7 through 9 shows the corresponding situation for support from above. The tube part 5 receiving the screw shank is connected rigidly to a shaped metal piece 24 which with a first wing 25 engages over the superior articular process and with a second wing 26 engages over the costal processed further wing 27 can be provided which engages under the superior articular process and/or under the costal process. The embodiments according to FIGS. 4 through 6 on the one hand and FIGS. 7 through 9 on the other hand can thus be combined in one shaped piece.

The illustrative embodiments according to FIGS. 1 through 10 show the tube part 5 on the convex side of the shaped piece 6 directed away from the bone. In the advantageous alternative embodiment according to FIG. 11, the tube part 5′ is arranged on the other, concave side of the shaped piece 6 directed toward the bone. Its external diameter tapers conically toward its end. Its length is chosen such that (measured parallel to the center line 1) it extends at least as far as the start of the supporting surface 7, preferably beyond this. Its total length is of the order of 1 to 1.5 cm. For receiving the tube part 5′, a bore hole of corresponding shape is formed in the bone. This bore hole can be slightly smaller in relation to the outer shape of the tube part 5′, so that, after the tube part 5′ has been inserted or hammered in, a secure fit is obtained, able to absorb forces.

The tube part configurations according to FIG. 10 and FIG. 11 can be combined with one another, as is shown in FIG. 12.

If the device according to the invention is intended. as a permanent implant, an intimate contact is desired between the tube part 5′, sitting in the bone, and the surrounding bone. For this purpose, the surface of this tube part 5′ can, as is indicated diagrammatically at 11, be provided with a surface configuration promoting intimate bone contact. For example, this can involve a roughened surface or porous coatings or bone-growth-promoting coatings, for example of hydroxyapatite.

The bone shapes in the area in question are extremely variable. It is therefore expedient to provide quite a large number of shaped pieces depending on the application requirements. It is also possible to assemble the pedicular screw in situ from a number of parts suitable for supporting at different sites, by means of corresponding connection devices, or at least to connect it to the screw shank in situ.

Support on the inferior articular process, which is indicated by reference number 9 in FIG. 1, can also take place within the scope of the invention.

1. An apparatus for stabilizing, alignment or fusion of vertebral bodies, comprising pedicular screws, rods connecting the screws, and a pedicular screw support configured to receive the pedicular screws and comprising a bore hole adapted to shanks of pedicular screws and at least one supporting surface which is designed for bearing from above or below on a costal process of the vertebral body, a superior articular process of the vertebral body, a saddle surface located on the vertebral body between the costal process and the superior articular process or on an inferior articular process of the vertebral body.

2. The apparatus as claimed in claim 1, wherein the supporting surface encloses an angle of less than 40° in the direction of the bore hole when viewed in sagittal section.

3. The apparatus as claimed in claim 1 or 2, wherein the bore hole is formed by a tube part connected to a shaped piece.

4. The apparatus as claimed in claim 3, wherein the supporting surface is formed by the concave side of the shaped piece and the tube part protrudes from the convex side of the shaped piece.

5. The apparatus as claimed in claim 3, wherein the tube part protrudes from the concave side of the shaped piece.

6. The apparatus as claimed in claim 5, wherein the tube part protruding from the concave side of the shaped piece is at least partially conical on the outside.

7. The apparatus as claimed in claim 5, wherein the tube part protrudes from the concave side at least as far as the supporting surface.

8. The apparatus as claimed in claim 5, wherein the outside of the tube part protruding from the concave side of the shaped piece has a configuration favoring connection to the bone.

9. The apparatus as claimed in claim 5, wherein the pedicular screw support is provided with a surface configuration promoting intimate bone contact.

10. The apparatus as claimed in claim 1 or 2, wherein the pedicular screw support is assigned in each case to only one pedicular screw.
